(19) United States
(12) Patent Application Publication
(10) Pub. No.: US 2007/0181131 A1
(43) Pub. Date: Aug. 9, 2007

(54) Ventilator safety coupling

(76) Inventor: William S. Lowery, Alameda, CA (US)

Correspondence Address: Bruce H. Johnsonbaugh;Eckhoff & Hoppe, Suite 2800, 101 Montgomery Street, San Francisco, CA, 94104, US

(21) Appl. No.: 11/701,027

(22) Filed: Feb. 1, 2007

Related U.S. Application Data
(60) Provisional application No. 60/771,129, filed on Feb. 7, 2006.

(51) Int. Cl. A62B 9/06; A61M 16/00

(52) U.S. Cl. 128/207.14

(57) ABSTRACT

A safety coupling is provided for use in a pulmonary pressure ventilator system to protect an intubated patient from adverse consequences of the ventilator tubing becoming disconnected from either an endotracheal tube or tracheotomy tube. The safety coupling is designed to assure continued airflow through the disconnected ventilator tubing, thereby assuring that the ventilator alarm that senses a disconnected tubing will operate properly. The safety coupling is simple in design with no moving parts. The coupling is either one or more openings formed at or near the proximal end of the ventilator tubing, and optionally may take the form of a tubular safety flange which is easily retrofitted onto existing pressure ventilators.

### CROSS-REFERENCE TO RELATED APPLICATION

This application claims the benefit of and priority from U.S. provisional application Ser. No. 60/771,129 filed Feb. 7, 2006.

### BACKGROUND AND BRIEF SUMMARY

The present invention relates generally to pulmonary ventilator safety devices. More particularly, the present invention provides a ventilator safety coupling which overcomes a potential hazard with presently available pressure ventilators.

The potential safety hazard with existing pulmonary pressure ventilators is that the coupling formed between the ventilator tubing and the distal end of an endotracheal (or tracheotomy) tube occasionally becomes separated. In this condition, the ventilator tubing connection to the endotracheal (or tracheotomy) tube is broken and typically an alarm would sound. Such alarms on ventilators are known as illustrated in the Isaza et al U.S. Pat. No. 6,668,824. Such disconnection can be caused by a vigorous cough by the patient or occasionally some malfunction with the ventilator. In some instances, when the ventilator tubing becomes separated from the distal end of the endotracheal tube, the proximal end of the ventilator tubing may contact a flat surface of the patient's body such as the patient's chest or neck area. Since the small proximal end of the ventilator tubing is smooth and flat, a seal may form between the surface of the patient's body and the ventilator tubing which may turn off the ventilator alarm and give a false indication that the pressure ventilator connection to the endotracheal tube remains intact. Needless to say, this particular situation can result in death, brain damage or other serious consequences.

The prior art does include a device addressing this problem referred to as a “Safe T-Tube,” shown and described in U.S. patent application Publication No. US 2003/0196666. The mechanism shown in that published application includes several movable parts and is unnecessarily complicated. The present invention overcomes the above-stated safety problem by providing a safety coupling which requires no moving parts and which is extremely simple in design.

A primary object of the invention is to provide a safety coupling for pressure ventilators having an extremely simple design with no moving parts.

A further object of the invention is to provide a safety coupling for pressure ventilators which can be easily retrofitted onto existing ventilators.

Other objects and advantages of the invention will become apparent from the following description and the drawings wherein:

### BRIEF DESCRIPTION OF THE DRAWINGS

FIG. 1A is a schematic representation of a patient having an endotracheal tube about to be connected to a pressure ventilator by a prior art connection;

FIG. 1B is a schematic representation of a patient having a tracheotomy tube about to be connected to a pressure ventilator by a prior art connection;

FIG. 2 is a schematic representation of the patient in FIG. 1A wherein the endotracheal tube is fully connected to the ventilator tube by a prior art coupling;

FIG. 3 illustrates a potentially fatal problem wherein the prior art ventilator tube has become disconnected from the endotracheal tube and has formed a seal with a portion of the patient's body;

FIG. 4 is a schematic illustration showing a front elevational view of one embodiment of the safety coupling of the present invention;

FIG. 5 is an exploded view of an optional form of the invention utilizing a tubular safety flange;

FIG. 6 is a schematic representation of the tubular safety flange shown in FIG. 5 as slidably engaging the proximal end of the ventilator tubing shown in FIG. 5; and

FIG. 7 is a schematic illustration of an alternate form of the invention wherein the openings are formed adjacent to the proximal end of the ventilator tubing.

### DETAILED DESCRIPTION OF THE DRAWINGS

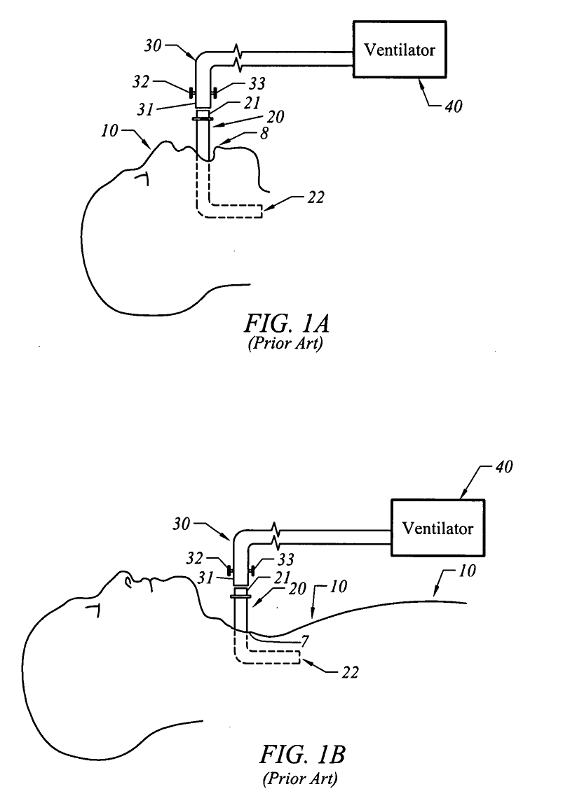

As shown in FIG. 1A, an endotracheal tube 20 is shown as inserted into the mouth 8 of an intubated patient 10. The distal end 21 extends upwardly away from the patient's mouth and the proximal end 22 (shown in phantom) extends into the trachea of the patient as is known in the art. A pressure ventilator shown generally as 40 includes various control mechanisms, displays and safety features, all of which are known in the art and are not described here for the sake of brevity. A ventilator tubing shown generally as 30 includes a proximal end 31 (relative to the patient) which forms a coupling with the distal end of endotracheal tube 21. A typical prior art coupling includes a pair of ears 32 and 33 carried by proximal end 31 of ventilator tubing 30. Ears 32 and 33 are typically attached by a rubber band to the endotracheal tube 20. As shown in FIG. 1A, the ventilator tubing 30 is ready to be connected to the endotracheal tube 20.

FIG. 1B illustrates the same pressure ventilator system 40 connected through the same tubing through a tracheotomy opening 7 in the throat of patient 10.

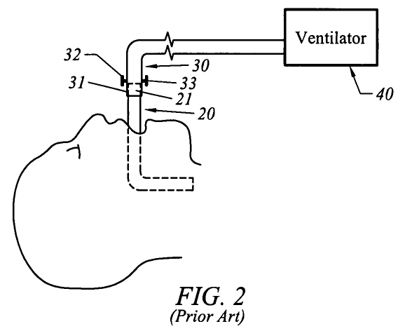

FIG. 2 shows the distal end 21 of endotracheal tube 20 inserted into the proximal end 31 of ventilator tubing by ears 32 and 33 being connected to the endotracheal tube 20 with rubber bands or other connectors (not shown) as is known in the art.

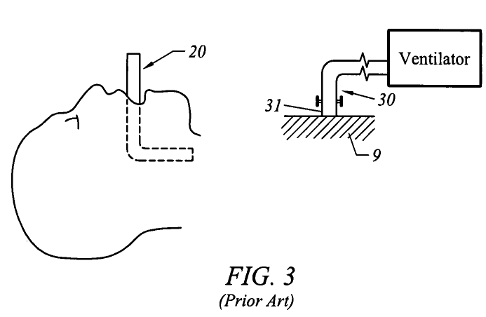

FIG. 3 illustrates the potential safety problem to which the invention relates. The ventilator tubing 30 has become disconnected from the endotracheal tube 20 and its proximal end 31 has formed a seal with a portion of the patient's body 9. In the position illustrated in FIG. 3, serious adverse consequences can occur very quickly, as noted above.

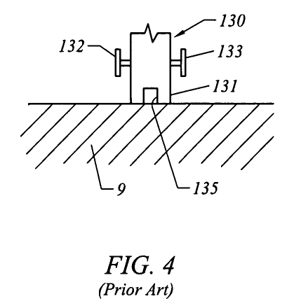

FIG. 4 illustrates a first embodiment of the invention wherein a modified ventilator tubing 130 is illustrated having coupling ears 132 and 133. The proximal end 131 of tubing 130 has a safety coupling means comprising at least a single rectangular airflow opening 135 (or preferably two rectangular openings) formed therein for the purpose of breaking a seal with the patient's body 9 which otherwise may occur as shown in FIG. 3. Airflow opening 135 allows continued flow so that the ventilator safety alarm will function. The word “airflow” is used broadly to include ambient air as well as pure oxygen and various combinations of gases used in ventilators. The shape of opening 135 may be other shapes such as curved or sawtooth shapes, provided that the area of the opening is sufficient to allow necessary airflow to maintain the ventilator alarms in an actuated state. In FIG. 4, the opening 135 is formed “at” proximal end 131, i.e., the opening 135 removes a portion of the periphery of proximal end 131.

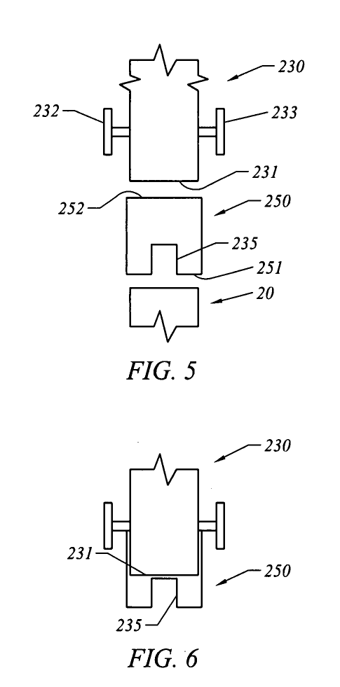

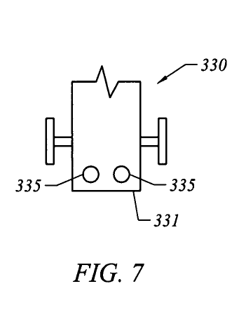

FIG. 5 illustrates an optional form of the invention wherein ventilator tubing 230 includes a flat smooth proximal end 231 and connection ears 232 and 233. In this embodiment, the safety coupling of the invention takes the form of a tubular safety flange 250, having a proximal end 251 and a distal end 252, which is a separate component that simply slides over the proximal end 231 of ventilator tubing 230 as shown in FIG. 6. At least one opening 235 is formed at the proximal end 251 of safety flange 250 for the same purpose described above and illustrated in FIG. 4. The advantage of the embodiment shown in FIG. 5 is that the safety flange 250 can readily be slid onto the proximal end 231 of an existing ventilator tubing line 230. Openings 235 of alternate shape may be formed in safety flange 250 such as curved shapes or sawtooth patterns. Openings 235 may be formed adjacent proximal end 251, similar to the openings shown below in FIG. 7.

FIG. 7 illustrates a further embodiment of the invention wherein ventilator tubing 330 has a smooth proximal end 331 and includes one or more openings 335 formed adjacent proximal end 331 provided that the area of openings 335 is sufficient to allow the necessary airflow as described above. Openings 335 do not extend through the smooth, flat proximal end 331 of tubing 330.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

What is claimed is:

1. In a pulmonary pressure ventilator system having a ventilator tubing, wherein said ventilator tubing has a distal end connected to said pressure ventilator system and a proximal end connected to an intubated patient through the distal end of either an endotracheal tube or tracheotomy tube, the improvement comprising:
safety coupling means carried by said proximal end of said ventilator tubing, said safety coupling means including one or more airflow openings to allow continued airflow through said opening or openings in the event said proximal end of said ventilator tubing becomes disconnected from either said endotracheal or tracheotomy tube.

2. The apparatus of claim 1 wherein said safety coupling means comprises one or more openings formed at said proximal end of said ventilator tubing.

3. The apparatus of claim 1 wherein said safety coupling means comprises one or more openings formed in said ventilator tubing adjacent said proximal end of said ventilator tubing.

4. The apparatus of claim 1 wherein said safety coupling means comprises a tubular safety flange adapted to slidably engage said proximal end of said ventilator tubing, said tubular safety flange having distal and proximal ends and having one or more openings formed either at or adjacent said proximal end.
